(19) United States
(12) Patent Application Publication
(10) Pub. No.: US 2015/0042954 A1
(43) Pub. Date: Feb. 12, 2015

(54) System and Method for Fluorescence Lifetime Imaging Aided by Adaptive Optics

(71) Applicants: Jennifer Hunter, Henrietta, NY (US); James Feeks, Rochester, NY (US)

(72) Inventors: Jennifer Hunter, Henrietta, NY (US); James Feeks, Rochester, NY (US)

(73) Assignee: University of Rochester, Rochester, NY (US)

(21) Appl. No.: 14/454,109

(22) Filed: Aug. 7, 2014

Related U.S. Application Data
(60) Provisional application No. 61/863,530, filed on Aug. 8, 2013.

(51) Int. Cl. A61B 3/12; A61B 3/113

(52) U.S. Cl. CPC A61B 3/1225; A61B 3/113
USPC 351/210; 351/246

(57) ABSTRACT

Techniques are illustrated herein for 1- and 2-photon fluorescence lifetime imaging in the living retina, using adaptive optics to correct aberrations and achieve cellular level resolution. 1-photon fluorescence embodiments may include the use of a confocal pinhole to provide axial sectiontin. 2-photon embodiments allow for inherent axial sectioning without having to block out-of-focus light.

### CROSS-REFERENCE TO RELATED APPLICATIONS

This application claims priority to U.S. Provisional Application No. 61/863,530, filed on Aug. 8, 2013, now pending, the disclosure of which is incorporated herein by reference in its entirety.

### FIELD OF THE DISCLOSURE

The disclosure relates to fluorescence lifetime imaging microscopy (“FLIM”) and ophthalmoscopy (“FLIO”).

### BACKGROUND

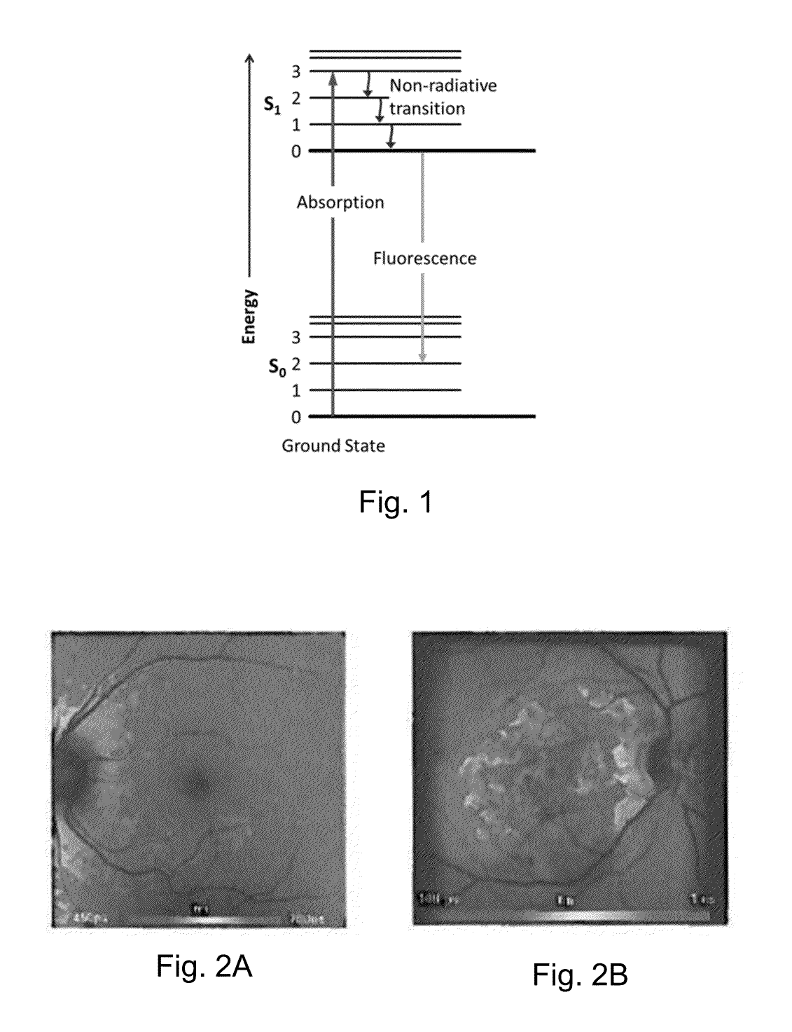

Fluorescence is a process wherein an electron within a molecule is excited to an upper electronic state (S1) by a photon (excitation photon). The molecule will relax to its lowest vibrational state within S1, and it will give off a photon (fluorescence photon) as it relaxes to its ground state (S0). The fluorescence photon will have lower energy than the excitation photon. See FIG. 1.

Many biologically important molecules fluoresce. Endogenous fluorophores include lipofuscin, nicotinamide adenine dinucleotide (“NADH”), flavin adenine dinucleotide (“FAD”), elastin, and collagen. Exogenous fluorophores, for example, fluorescein and green fluorescent protein (“GFP”), can be used in dyes to label cells. By analyzing the intensity, excitation and/or emission spectrum, lifetime, or anisotropy of the fluorescence signal, it is possible to deduce information about a cell.

When a population of atoms or molecules is excited by light, the number of molecules N in the excited state decays as:

$$\frac{d N ( t )}{d t} = - ( Γ + k ) N ( t )$$

$$N ( t ) = {N}_{0} {e}^{- ( Γ + k ) t} = {N}_{0} {e}^{- t / τ}$$

where Γ is the radiative decay rate (emission of photons), k is the non radiative decay rate (collisions with other molecules, etc.), and τ is the “fluorescence lifetime,”—the time it takes for the fluorescence intensity to drop off to 1/e of its maximum value.

Fluorescence lifetime is useful for measuring intra- or intercellular environmental parameters such as: ion concentration by fluorescence quenching, oxygen levels by fluorescence quenching and/or “redox ratio,” cellular metabolism (through autofluorescence of the coenzymes NADH and FAD), Förster Resonance Energy Transfer (“FRET”) which manifests as a reduction in lifetime of the donor molecule due to energy transfer to an acceptor (useful for investigating protein interactions and molecular distances within cells).

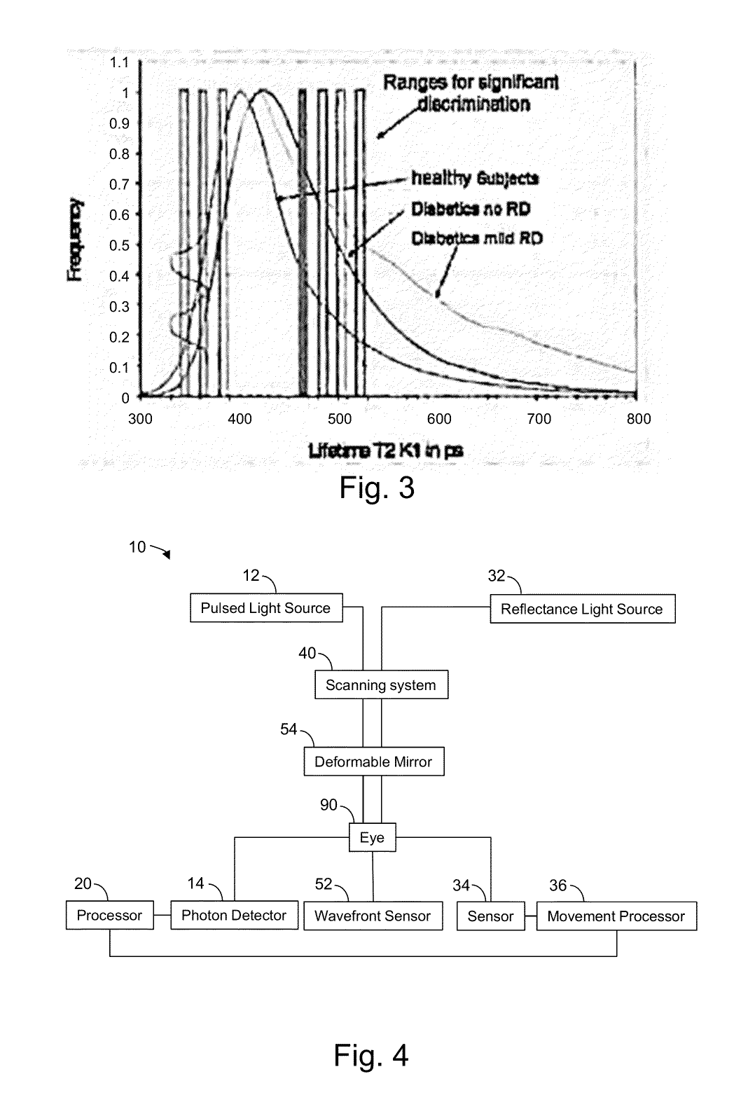

Fluorescence lifetime imaging microscopy (“FLIM”) has been used in the living eye to image a patient with advanced AMD (FIG. 2B) and show the differences from FLIM images of a normal eye (FIG. 2A). FLIM has also been used to measure early pathologic changes in diabetic retinopathy, before structural signs are visible (FIG. 3). 2-photon FLIM has seen both clinical and research use in, for example, melanoma detection, cosmetics research, drug monitoring, measuring the efficacy of drug therapy on breast cancer tumors in rodent.

There is a need for FLIM capabilities having enhanced resolution (e.g., single cell) and the use of FLIM using 2-photon excitation in the eye to provide axial sectioning and the ability to better excite NADH and FAD.

### BRIEF SUMMARY

According to aspects illustrated herein, there is provided an apparatus and methods for 1- and 2-photon fluorescence lifetime imaging in the living retina, using adaptive optics to correct aberrations and achieve cellular level resolution. 1-photon fluorescence embodiments may include the use of a confocal pinhole to provide axial sectiontin. 2-photon embodiments allow for inherent axial sectioning without having to block out-of-focus light, reduced photobleaching of fluorophores, and the ability to excite NADH and FAD maximally due to 2-photon effect (whereas single photon excitation of these molecules is largely blocked by the optics of the eye).

Embodiments of the present disclosure may be useful for characterization of lipofuscin deposits, measurement of functional metabolic state of various retinal layers by measuring lifetimes of NADH, FAD (both in bound and free states) in conjunction with redox ratio of NADH/FAD, diagnosing and interrogating retinal disease at the cellular level (changes in free versus bound NADH in certain diseases), and measuring drug or therapeutic efficacy by interrogating the same region at intervals during therapy administration, arterial (or capillary) occlusion causing change in metabolic activity and change in pH. Additionally, functional measurements of retinal activity may be accomplished by, for example, stimulating certain photoreceptors and measuring the metabolic response on either the photoreceptors or ganglion cells. Retinol and retinoids of the visual cycle can also be useful as possible markers of interest.

Description of the Drawings

For a fuller understanding of the nature and objects of the disclosure, reference should be made to the following detailed description taken in conjunction with the accompanying drawings, in which:

FIG. 1 is a graphic depicting energy states of an electron bound to a molecule;

FIG. 2A is a FLIM image of a normal eye;

FIG. 2B is a FLIM image of the eye of a patient with advanced AMD;

FIG. 3 is a chart showing the use of FLIM to distinguish diabetic retinopathy;

FIG. 4 is a block diagram of an apparatus according to an embodiment of the present disclosure; and

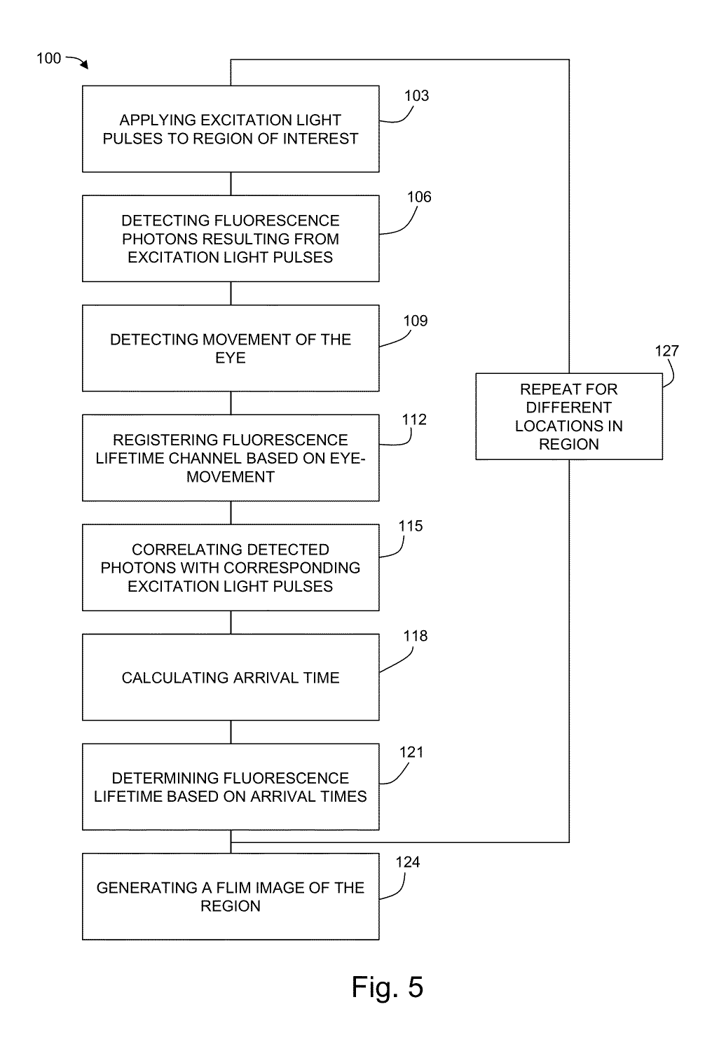

FIG. 5 is a flowchart depicting a method according to another embodiment of the present disclosure.

### DETAILED DESCRIPTION

The present disclosure may be embodied as an apparatus 10 for in vivo fluorescence lifetime imaging microscopy of a region of interest of an eye 90, such as, for example, the retina or a portion thereof. The apparatus 10 may be considered a modified adaptive optics scanning laser opthalmoscope (“AOSLO”).

The apparatus 10 comprises a pulsed light source 12, for providing excitation energy to the region. The pulsed light source 12 may be, for example, a picosecond laser. Other pulsed light sources 12 may be used, and further examples are provided below with descriptions of single-photon and 2-photon fluorescence. The pulsed light source 12 is used to excite a focal area of the region of interest—a location of the region where the light source is focused—with a plurality of light pulses. The light pulses may cause fluorescence in the focal area.

The apparatus 10 includes a photon detector 14 for detecting the photons resulting from fluorescence within the focal area. The photon detector 14 may be a low-noise detector suitable for detecting single photons, such as, for example, a photomultiplier tube, a hybrid photomultiplier tube, a single photon avalanche diode (“SPAD”), or other suitable detectors. The photon detector 14 generates an electrical signal corresponding to detection of photons.

A processor 20 is in electrical communication with the pulsed light source 12 and the photon detector 14. The processor 20 receives electrical signals from the photon detector 14 and can determine a plurality of arrival times, each arrival time being the elapsed time between a light pulse and its corresponding fluorescence photon. An arrival time may be determined by detecting a pulse and determining how long until the corresponding fluorescence arrives. In another embodiment, fluorescence is detected and the time to the next pulse of light is determined in order to back-calculate the arrival time (knowing the repetition rate of the laser). The plurality of arrival times may be used to generate a histogram such that the arrival time data for the focal area may be analyzed. An exemplary processor 20 is a time-correlated single photon counting module (“TCSPC”) such as those available from Becker & Hickl.

The apparatus 10 further comprises a reflectance imaging system 30 used to detect movement of the eye 90. The reflectance imaging system 30 is configured to detect such eye 90 movement and generate an eye-movement signal (tracking signal). In an exemplary embodiment, the reflectance imaging system 30 has a light source 32, a sensor 34 capable of capturing high-SNR reflectance images of at least a portion of the region of interest, and a movement processor 36 in electrical communication with the sensor 34. The sensor 34 may be used to capture two or more images of the region of interest over a time interval. The movement processor 36 may then determine eye movement by comparison of the captured images. The movement processor 36 can then generate an eye-movement signal based on the determined eye movement. Other eye-tracking systems may be used to generate an eye-movement signal and are considered within the scope of the present disclosure.

The processor 20 is in electrical communication with the reflectance imaging system 30. In order to correlate the detected fluorescence photons with the corresponding light pulses, the processor 20 may receive and use the eye-movement signal to compensate for movement of the eye (i.e., register the detected photons in the fluorescence lifetime channel). Separate processors may be used for the TCSPC and registration functions. In an embodiment, modifications can be made to existing image registration software in order to properly register the photons detected in the fluorescence lifetime channel. This will involve exporting the photon counts from fluorescence lifetime software (of, for example, a TCSPC), co-registering with the high-SNR reflectance image, binning the photons in the proper pixel and time, and feeding this data back into the fluorescence lifetime software for additional processing.

An exemplary embodiment of an apparatus according to the present disclosure may be used for single-photon fluorescence. In such an embodiment, it is known in the art to use techniques such as confocal microscopy to detect fluorescence occurring at the desired focal plane (i.e., depth within the retina—axial sectioning).

In another embodiment of the presently disclosed apparatus, the apparatus may be used for 2-photon fluorescence microscopy. Such 2-photon systems are known as providing inherent axial sectioning due to the conditions for 2-photon fluorescence being present substantially at only the focal plane. In such a 2-photon embodiment, the pulsed light source may be, for example, a titanium:sapphire laser.

The apparatus 10 may further comprise a scanning system 40 configured to change the location of the focal area within the region of interest. For example, the apparatus 10 may comprise pivoting and/or rotating mirrors for scanning in the x- and y-directions and moveable optics for changing the focal plane (z-direction). The scanning system 40 may be in electrical communication with the reflectance imaging system 30 such that the scanning system 40 can adjust and compensate for eye-movement. Scanning rates up to 8 kHz may be used and higher scanning rates, ranging to 16 kHz or higher, may be used to reduce the effect of eye-motion, thereby improving accuracy.

An apparatus 10 of the present disclosure may further comprise an adaptive optical system 50 configured to adjust to changes and/or aberrations in the optics of the eye. In some embodiments, an adaptive optical system 50 may comprise a wavefront sensor 52, such as, for example, a Shack-Hartmann wavefront sensor, for detecting the shape (i.e., local tilt) of a wavefront. A mirror 54, such as, for example, a MEMS deformable mirror, may be used to compensate for the detected changes/aberrations. The processor 20 may be in electrical communication with the adaptive optical system 50 in order to compensate for changes when correlating fluorescence photons with light pulses. Other forms of adaptive optics are known and within the scope of the present disclosure, including, without limitation, the use of spatial light modulators for correction.

The present disclosure may be embodied as a method 100 for in vivo fluorescence lifetime imaging microscopy of a region of interest of an eye, such as, for example, the retina.

The method 100 comprises the step of applying 103 a plurality of excitation light pulses to a first location of the region of interest. Such excitation light pulses may be applied 103 using, for example, a pulsed picosecond laser. The method 100 further comprises detecting 106 fluorescence photons resulting from the applied 103 excitation light pulses.

The method 100 comprises the step of detecting 109 movement of the eye (e.g., detecting 109 movement of the region of interest of the eye). The detected 109 eye movement is used to register 112 the detected photons 106 with the corresponding applied 103 excitation light pulses. Each detected 106 photon is correlated 115 with a corresponding applied 103 excitation light pulse.

Having correlated 115 the detected 106 photons with corresponding excitation light pulses, an arrival time for each photon may be calculated 118. The method 100 may further comprise the step of determining 121 a fluorescence lifetime value for the first location based on the calculated 118 arrival times. For example, the arrival times may be binned and a lifetime value may be determined 121 statistically on the binned arrival times.

Each step of the method 100 may be repeated for a plurality of locations of the region of interest. For example, a 2- or 3-dimensional array of positions may be scanned (e.g., rastered) to determine 121 fluorescence lifetime value for each positon in the array. These determined 121 lifetime values may be used to generate 124 an image of the region of interest wherein each fluorescence lifetime value is used as the value of a corresponding pixel of the image (for example, each location of the plurality of locations corresponds to a pixel of the image).

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims. The claims can encompass embodiments in hardware, software, or a combination thereof

What is claimed is:

1. An apparatus for in vivo fluorescence lifetime imaging microscopy of a region of interest of an eye, comprising:
a reflectance imaging system configured to detect movement of the eye and generate an eye-movement signal;
a pulsed light source for exciting a focal area of the region of interest with a plurality of light pulses;
a photon detector for detecting fluorescence photons resulting from excitation of the focal area, the photon detector configured to generate an electrical signal corresponding to detection of a photon; and
a processor in electrical communication with the photon detector, the light source, and the reflectance imaging system, the counter configured to:
correlate the detected fluorescence photons with the corresponding excitation light pulses and using the eye-movement signal to account for movement of the eye; and
calculate the arrival time as the elapsed time from the application of excitation light to the detection of the corresponding fluorescence photon.

2. The apparatus of claim 1, wherein the pulsed light source is a picosecond pulsed laser.

3. The apparatus of claim 1, wherein the pulsed light source is a femtosecond laser.

4. The apparatus of claim 1, wherein the pulsed light source is configured for single-photon excitation of the region of interest.

5. The apparatus of claim 1, wherein the pulsed light source is configured for 2-photon excitation of the region of interest.

6. The apparatus of claim 5, wherein the pulsed light source is a titanium:sapphire laser.

7. The apparatus of claim 1, wherein the photon detector is a hybrid photomultiplier tube, a photomultiplier tube, or a single photon avalanche diode.

8. The apparatus of claim 1, further comprising a scanning system configured to change the position of the focal area within the region of interest.

9. The apparatus of claim 1, wherein the reflectance imaging system is configured to:
obtaining a first image of a portion of the region of interest;
obtaining a second image of the portion of the region of interest;
determining movement of the eye based upon the differences between the first image and the second image.

10. The apparatus of claim 1, further comprising an adaptive optical system configured to adjust to changes in the eye and the excitation light pulses and fluorescence photons are transmitted by way of the adaptive optical system.

11. The apparatus of claim 10, wherein the adaptive optical system includes a wavefront sensor and/or a deformable mirror.

12. A method for in vivo fluorescence lifetime imaging microscopy of a region of interest of an eye, comprising:
applying a plurality of excitation light pulses to a first location of the region of interest;
detecting movement of the eye;
detecting photons resulting from fluorescence caused by the excitation light pulses;
registering detected photons to excitation light pulses based on the detected eye movement;
correlating each detected photon with the corresponding excitation light pulse; and
calculating an arrival time for each detected photon.

13. The method of claim 12, further comprising determining a fluorescence lifetime value for the first location based upon the calculated arrival times.

14. The method of claim 13, further comprising repeating each step for a plurality of locations of the region of interest.

15. The method of claim 14, further comprising generating an image representation of the region of interest based on the determined fluorescence lifetime value for each of the locations of the region of interest.
